(19) United States
(12) Patent Application Publication
(10) Pub. No.: US 2019/0386610 A1
(43) Pub. Date: Dec. 19, 2019

(54) SUSTAINABLE ENERGY PRODUCTION

(71) Applicant: Thomas Regrut, Westerville, OH (US)

(72) Inventor: Thomas Regrut, Westerville, OH (US)

(21) Appl. No.: 16/092,316

(22) PCT Filed: Mar. 5, 2018
(86) PCT No.: PCT/US18/20904

§ 371 Date: Oct. 9, 2018

Related U.S. Application Data
(60) Provisional application No. 62/467,556, filed on Mar. 6, 2017.
(60) Provisional application No. 62/570,709, filed on Oct. 11, 2017.

(51) Int. Cl. H02S 40/44; H02S 20/22; H02S 40/22; F24S 20/40; F24S 20/60; C12P 7/08; C12F 3/02

(52) U.S. Cl. CPC H02S 40/44; H02S 20/22; H02S 40/22; C12F 3/02; F24S 20/60; C12P 7/08; F24S 20/40

(57) ABSTRACT

To reduce the overall environmental impact of a residence or commercial facility, a system is provided for integrating energy usage. In such a system, there is a system for capturing solar energy; a system for fermenting biomass and concentrating the fermentate, generating carbon dioxide and ethanol; a system for storing excess energy for subsequent release; and a system for growing biomass. In integrating the systems, the captured solar energy is used as heat and electrical power. Excess energy beyond an instantaneous energy requirement is stored in the system for storing excess energy. Ethanol produced is used as a fuel. Carbon dioxide that is produced is provided to the system for growing biomass. Instantaneous energy deficiencies are reduced by releasing stored excess energy.

### TECHNICAL FIELD

The embodiments disclosed herein relate to modifications and adaptations that are made to a pre-existent household, as well as to new constructions, for the purpose of reducing the energy footprint of the residents and the residence as well as an existing or future electricity generation facility. In addition, a scalable energy storage and electrical generation system is disclosed herein for a wide range of possible applications, from a household size all the way up to utility size for both renewable energy sources as well as traditional energy sources. Finally, a new construction method is disclosed herein which provides an optimal, attractive roof for the installation of photovoltaic cells and solar thermal equipment in the attic of homes or other types of buildings, providing a previously unused space the opportunity to contribute to the energy efficiency of the household or other type of building.

### BACKGROUND OF THE ART

Much discussion has occurred lately over the issue of global warming or climate change. Also, there is still a great deal of discussion and debate concerning the question of whether it is due to human sources. Regardless of the source, it is difficult to challenge the increases in atmospheric carbon dioxide and the increased levels of the carbon dioxide into the oceans. Also, glacial retreat and its effects on global sea levels since the beginning of the industrial revolution are well-documented.

The retreating glaciers and the increased carbon dioxide levels in the oceans are the proverbial canaries in the coal mine. While many programs have focused on the impact of carbon dioxide, many causes have all played a role in climate change. These include several natural phenomena, such as volcanic eruptions, ocean currents, variances in the earth's orbit and solar cycles. These provide little to no opportunity for human intervention. Some causes are partially natural and partially man-made, such as desertification and deforestation, each of which provides some opportunity for human intervention. Lastly, greenhouse gas emissions and fossil fuel heat emissions are largely man-made and provide the best opportunity for human intervention.

When greenhouse gases are mentioned, carbon dioxide receives a great deal of attention, yet other greenhouse gases pose a similar and perhaps greater threat. These include carbon dioxide, methane, halocarbons and nitrogen oxides. Of these, the halocarbons, which are all substantially man-made, pose a very long-lasting threat. The combined effects of all of these causes all play a role in climate change. An effort is needed to eliminate or mitigate any man-made factor that has a negative impact on the environment and that has a negative impact on climate change.

It becomes clear that efforts are needed to reduce man-made emissions of carbon dioxide, methane, halocarbons and nitrogen oxides. Waste heat emissions must also be targeted. Reducing deforestation would include reducing wildfires, with the obvious effects on carbon dioxide.

Some “back of the envelope” calculation provides some insights into the magnitude of the impact of human activity on climate change. First, addressing the issue of carbon dioxide emissions, the composition of greenhouse gas emissions, on a dry basis, is approximately 72% carbon dioxide, 18% methane (uncombusted) and 9% nitrogen oxides. Since the onset of the Industrial Revolution in the early 1800s, global carbon dioxide levels have increased from about 100 ppm to about 400 ppm.

Some rough numbers indicate the magnitude of the problem just in terms of the role of waste heat which is typically not included in climate change models because the role of waste heat is so small. Based on an annual 27 billion metric tons (tonnes) of carbon dioxide emissions (2005 estimate), a calculation can be made. Assuming that all carbon dioxide results from methane oxidation (a reaction that yields 212 kcal per mole), waste heat can be estimated.

Using known conversion factors, it is seen that 27 billion tonnes of carbon dioxide represents 6.14×10¹⁴ moles of carbon dioxide. Using the 212 kcal per mole recited above, the heat released annually to supply the carbon dioxide amounts to 1.30×10¹⁷ kcal.

How much energy is this? In terms that can be understood, the energy release of the atomic bomb dropped on Hiroshima in 1945 has been estimated at 15 kilotonnes of TNT, where 1 tonne of TNT has an energy value of 999,300 kcal. On that basis, the waste heat released in 2005 as a result of carbon dioxide production amounts to 1.30 x 10¹¹ tonnes of TNT. When divided by the 15,000 tonnes for a Hiroshima-sized bomb, the result of 8.68 million Hiroshima-sized atom bomb blasts per year is obtained. This is approximately 23,800 such blasts per day! That is very sobering, bad news; however, the full magnitude of the problem is far worse. There are estimates for the role of waste heat being in the range of 1% to 6% of the role of the greenhouse effect. Being an optimist, let us assume for the following calculation that the role of waste heat is 5%. This assumption allows the math to remain simple; the daily waste heat needs to be multiplied by 19 to estimate the role of recycling the radiation due to greenhouse gases. On that basis, heat release amounts to 23,800 Hiroshima atomic bomb blasts per day and greenhouse gases amount to 452,200 Hiroshima atomic bomb blasts per day, for a total of 476,000 Hiroshima atomic bomb blasts per day.

This is a very serious problem, which will only grow even more serious the longer that essentially nothing is done to address this problem, with the exception of endless debate on the topic of whether or not a problem exists. The paralysis that has arisen due to this endless, charged debate has resulted in a lack of action to address this problem in a coordinated, effective manner.

The scientific principles that conclusively support the contention that greenhouse gases cause an increase in the amount of energy retained by the earth are actually quite simple and irrefutable. If anyone ever went outside as a child and played in the rain, the simple act of making a dam in a gutter during a rainstorm provides an analogy to what is happening in the atmosphere. So, what actually happens when you go outside in the rain and make a dam in the gutter? The amount of upstream water increased significantly. This same effect is occurring on our planet. It is well known and widely understood that greenhouse gases cause infrared radiation that would normally escape into outer space to be reflected (recycled) back to the earth repeatedly. Thus, the greenhouse gases in the atmosphere present the same obstacle to flow that the dam presented during the rainstorm. The heat backs up. The heat accumulates. The accumulation of heat increases the temperatures of the ground, the oceans, and the atmosphere. The accumulation of heat also causes glacial ice to melt.

Two observations of glacial ice indicate the presence of the proverbial canary in the coal mine. Arctic Sea Ice currently melts almost completely in the summer months. This is a phenomenon that is new that has not been experienced for quite a long time, perhaps 2 millennia. Glacier National Park in the United States has seen such dramatic decline in glaciers that the park has a predicted future of being ice-free by sometime in the 2030's. Please reflect on the fact that a National Park with Glaciers will no longer have any ice; this outcome is eerily similar to the Cuyahoga River catching on fire in the state of Ohio.

These compelling observations, along with the shockingly high level of the impact of heat being added to the planet daily, 476,000 Hiroshima atomic bomb blasts per day, demand an immediate, effective, feasible solution to this massive problem. Further delays will only make the final day of reckoning yet more disastrous. Simply put, business cannot continue as usual, with an endless debate. If we debate until the oceans boil, we will certainly have debated far too long. The scientific community needs to step forward and come to a consensus quickly in order to speak with one voice to a world that is trapped in an endless political debate. After that, it is time to take action to deal with a problem that actually has a solution that will improve the quality of life in this world for a significant number of people in our society with just a different lifestyle in terms of our collective attitude and behavior with respect to energy use.

It is therefore an unmet advantage of the prior art to provide an energy model and system for a typical household to reduce its waste heat while reducing fossil fuel consumption as well as provide an energy model and system for a typical electricity generation facility to reduce its waste heat while reducing fossil fuel consumption. It is therefore also an unmet advantage of the prior art to provide an energy storage and electrical generation system that is reusable, highly sustainable, environmentally-friendly, available in more than adequate supply throughout the word at no cost, and scalable. It is also therefore an unmet advantage of the prior art to provide a new method to construct an attractive, durable roof that offers an optimal location to install photovoltaic cells and/or solar thermal collection equipment in existing and new homes and buildings. It is also therefore an unmet advantage of the prior art to provide a wide range of new configurations of appliances, electrical switches, equipment, and other assorted items to take advantage of the new capability that will be installed in homes and buildings to harvest and store renewable energy.

### SUMMARY OF THE INVENTION

These and other unmet advantages are provided by a system for integrating energy usage. Such a system comprises a system for capturing solar energy, a system for fermenting biomass and distilling the fermentate, generating carbon dioxide and ethanol, a system for storing excess energy for subsequent release; and a system for growing biomass, operating in an integrated manner in which the solar energy captured is used as heat and electrical power, with any excess energy stored in the system for storing excess energy. The ethanol is used as a fuel, carbon dioxide is provided to the system for growing biomass, and instantaneous energy deficiencies are reduced by releasing stored excess energy.

In some embodiments, the system for capturing solar energy comprises a thermal transfer fluid circulated in tubes. Particular embodiments of that system further comprise reflectors to focus the solar energy onto the tubes

In other embodiments, the system for capturing solar energy comprises photovoltaic cells generating electrical energy.

In either the thermal transfer case or the photovoltaic case, the system for capturing solar energy is preferably arranged inside an attic space of an inhabitable dwelling.

In some embodiments, the system for storing excess energy comprises a compressor, powered by excess energy from the system for capturing solar energy solar to compress a fluid, at least one storage means for containing the compressed fluid, a an expander for expanding compressed fluid from the at least one storage means. The power generated by the expander is used to reduce instantaneous energy deficiencies in the system. In other embodiments, the system for storing excess energy comprises a gas liquefaction unit instead of a compressor. In many of these embodiments, the fluid compressed is air, and the gases, when liquified, may be separated into components.

### BRIEF DESCRIPTION OF THE DRAWINGS

Some aspects of the invention will be better understood when reference is made to the attached drawings, wherein identical parts are identified with identical reference number and wherein:

FIG. 1 shows a schematic illustration of a first solar thermal application of the inventive concepts;

FIG. 2 shows a schematic illustration of a second solar thermal application of the inventive concepts;

FIG. 3 shows a schematic representation of a system for residential alcohol production; and

FIG. 4 shows a schematic illustration of an inventive concept for storing energy generated.

### DETAILED DESCRIPTION OF A PREFERRED EMBODIMENT

Viewed initially from a high level, the disclosed embodiments relate to methods involving the production of electricity using an invention which the inventor has entitled a hybrid clean energy unit, and the use of waste heat at the electrical energy generation site to drive any appropriate endothermic process, including but not limited to, ethanol fermentation, conversion of waste plastic to fuel, coal liquefaction, or coal gasification. In addition, the waste heat may be used to effect a physical transformation or purification method, such as the distillation of sea water so that it could be used as potable water.

### Production of Electricity from Fossil Fuels

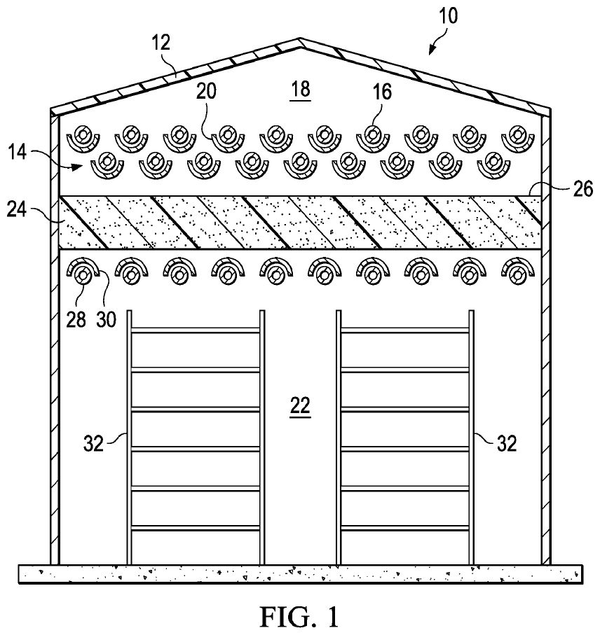

FIG. 1 illustrates schematically how a solar thermal application can be used to reduce electricity or natural gas usage. In the embodiment 10, a transparent or translucent roof 12 is used instead of a typical opaque roof. By doing this, the solar thermal apparatus 14, depicted as a series of tubes 16, is located inside the housing, in an attic space 18 in the depicted embodiment. This eliminates the need to be outside on a roof surface. In this embodiment, an appropriate heat transfer liquid (water being a good example) passes through the tubes 16 that are located in an attic space 18, acquiring heat in the process. An optional set of reflectors 20 is shown below each of the tubes 16. In a preferred embodiment, these reflectors 20 are mechanized to track the movement of the sun. The attic space 18 is separated from an inhabitable space 22 by an insulative layer 24. In addition to, or possibly instead of, the surface 26 that faces into the attic space 18 may be coated with a reflective material. In some embodiments, only a single tube 16 may be used.

Below the insulative layer 24, a series of tubes 28 circulate the heated heat transfer fluid during heating seasons, before the heat is extracted for other purposes. These tubes 28 are also shown with optional, but preferred, reflectors 30. In a non-heating portion of the year, heated fluid in tubes 16 is shunted directly for heat extraction. In some situations, it may be desirable to use a clear polymeric sheet, such as a polycarbonate or a poly(methyl methacrylate), as the insulative layer 24. By doing this, the solar energy incident on the upper surface will either be reflected or refracted through the layer. This allows the inhabitable space 22 to be naturally lighted during daylight hours. In some embodiments, the inhabitable space will instead find use as a greenhouse, with the installation of grow racks 32. When this solar thermal embodiment 10 is used with a fermentor or another source of carbon dioxide, a greenhouse space such as this can be provided with an enhanced carbon dioxide level, to facilitate growth. Of course, in such a case, it would be prudent to monitor carbon dioxide levels in the inhabitable space, as a safety procedure.

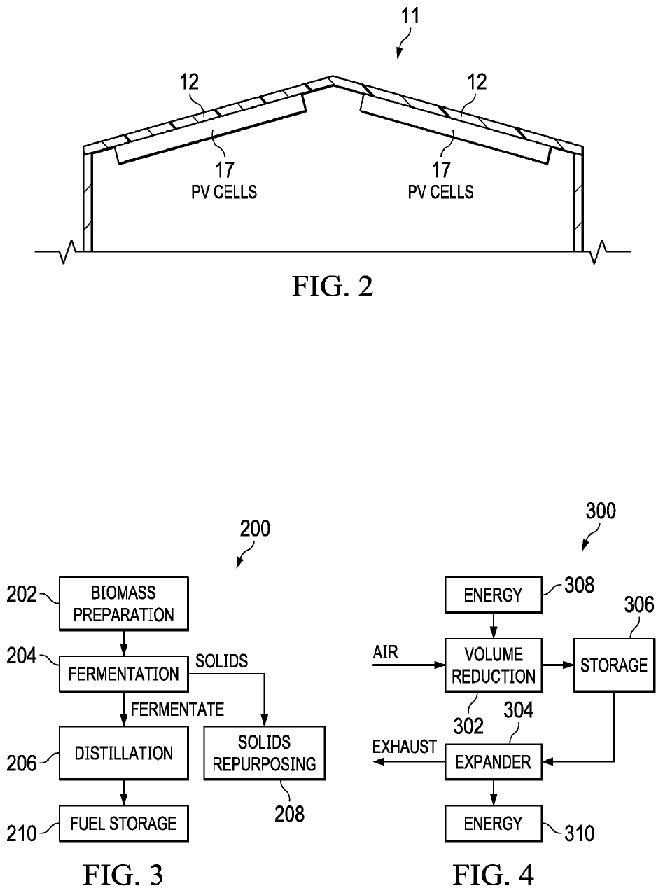

In another embodiment 11, as depicted in FIG. 2, a series of photovoltaic panels 17 are deployed, preferably immediately below and parallel to the roof surface 12. In this embodiment 11, the electricity produced in the photovoltaic panels 17 substitutes for electricity that would otherwise be drawn from the overall electrical grid.

These hybrid approaches to renewable energy are available in the middle latitudes of the United States and may be extendable to other geographical areas with similar seasonal climates through the deployment of the invention, a hybrid clean energy unit. From April through November, a solar thermal array can be configured at a residence or at an electricity generation facility. The array can be arranged similarly to those already in use, with the solar energy being used to preheat and/or boil water prior to being sent to a boiler, among other uses, reducing the need for fuel. In preferred embodiments, the solar collection would be by way of parabolic mirrors that are automated to follow the sun's position throughout the day. In the cold months, typically, November through April, exhaust waste heat from the electric plant will be used for space heating. To do this, the waste heat emissions from the stack will be passed through the upper level of the space in a pipe that would be surrounded by a reflector element to radiate the heat to the space below. As the need arises, the space heated by the waste heat may also be supplemented by radiant heat generators at floor level. In addition, the space below could be utilized for any number of possible uses.

One of the possible uses is biomass growth, using greenhouse techniques, during the winter season. The heat from the power plant would be used to essentially provide a space for this endeavor. This approach would work so long as the power plant was capable of providing a consistent level of heat to maintain the temperature in the greenhouse space for the plants. In addition, the greenhouse space could be used in the warmer months provided that sufficient shielding and insulation is provided when the solar energy is being harvested. In addition, windows could be added to the greenhouse just below the shielding and insulation, or in other places, to ventilate the greenhouse as the need arises.

One of the other possible uses of the area below the solar thermal array is an area for livestock. Livestock may live in this area a high portion of their life. With this arrangement, the methane produced by Livestock is actually contained and sent to a conventional carbon and/or hydrocarbon and/or biomass and/or ethanol and/or fuel oil electrical generation facility where it fed to a boiler and/or generator to produce electricity. In addition, this unit is designed such that manure from the livestock falls through the grates where it may be easily collected and used for a number of purposes, including but not limited to, anaerobic digestion, fertilizer, and/or composting.

This hybrid approach to renewable energy helps the electric generator move toward sustainability on a number of fronts. First, fossil fuel use for electric generation is reduced in the warm months via the use of solar thermal. Second, waste heat from the power plant is reduced in the cold months via the use of the waste heat from the electric power plant for the space heating for the greenhouse space or other use of the space. Third, vegetables, lettuce greens, and fruit and other plants can be produced in the winter months and summer months for consumption. Fourth, if crops can be grown closer to the customer, this would yield a savings in transportation fuel needed as opposed to transportation of crops grown far away. Fifth, the methane from livestock may now be harvested and combusted to produce electricity. Sixth, the manure from livestock may now be collected and used for a productive purpose. In addition, this type of growing condition helps the farmer to not have to be at the mercy of the weather for the success of the crops. In addition, livestock do not have to be at the mercy of the weather either, for heating and ventilation can be provided to maintain a more comfortable living area for livestock. Another possible benefit is that biomass from these operations could be burned for electric generation.

Because plants utilize carbon dioxide, providing an area of enhanced carbon dioxide concentration in the growing area will increase the rate of plant growth. It is known that carbon dioxide, having a molecular weight significantly higher than oxygen, nitrogen or air, which is a mixture of approximately 80% nitrogen and 20% oxygen, will tend to stratify out below the air into the lower portion of a space, unless the gas in the space is stirred or moved to prevent the stratification. Locating the plants near the floor will allow workers to operate in the space, in spite of the higher carbon dioxide level.

It is also important to note that an Energy Storage and Electrical Generation System may be added to any of the foregoing inventions to provide an electrical power back up for the facility in the event that the primary source of power experiences a shutdown. The Energy Storage and Electrical Generation System is highly scalable and very flexible for any application. In addition, the material used for this equipment is air. Air is reusable, highly sustainable, environmentally-friendly, and available in more than adequate supply throughout the word at no cost.

This method addresses the dual problem of glacial melting and increasing carbon dioxide levels. Through the use of this method and system, waste heat is reduced and the amount of carbon dioxide generated for a given unit of electricity has been decreased. In short, waste heat has been reduced and less fossil fuel is being used. In addition, a source of fresh produce is available in the winter months much closer to the marketplace.

The inventor envisions the manufacture of these hybrid clean energy units to be done remote from the point of use, to which a pre-fabricated system would be delivered. Ideally, the units could be sized so that they can be transported to the electric generation facility or farm by rail or truck. Additionally, the use of an assembly line construction method or a cell construction method would support higher productivity, more consistent fabrication quality, cost control, and more oversight of the work being completed.

Another option for an electric generator or farmer is to provide waste heat on an annual basis for space heating needs and hot water needs. In the winter, waste heat can provide space heat to those in need of it. In the summer months, the steam can drive an absorption cooler to provide air conditioning.

Another alternative to reduce the coal required for electricity lies in simply combusting grass clippings and/or biomass. Presently, some grass clippings are composted; however, some grass clippings simply go to the landfill. The combustion of grass clippings presents a problem for electricity generators due to the chemical composition of the grass clippings. Chromium deposits on boiler equipment present an unwelcome problem for electricity generators that arises from burning biomass such as grass clippings. In a residential setting, the solar grass demineralizer would be set up on the sunniest acceptable side of the home. Water that would normally be going to the water heater would be routed through the solar grass demineralizer in piping. Fresh grass clippings would be placed in the top of the unit. As the clippings are exposed to sunlight and compost, heat will be generated. This heat would be transferred through the walls of the pipe to the water headed to the water heater. After sufficient residence time in the solar grass demineralizer, the grass clippings would break down and demineralize. The grass clippings would then be dropped out of the bottom of the unit either into bags to be sent to waste collection or directly onto the ground for further demineralization and subsequent bagging for waste collection or for use by the homeowner as a soil amendment. Grass clippings that are bagged and picked up by waste collection would then be briquetted using existing process technology. After briquetting, the grass briquettes could then be combusted alongside coal in the boiler. Since these grass briquettes have been properly demineralized, the chromium deposit problem in the boiler has been addressed and should not present a problem. The benefits of this practice are numerous. First, a carbon neutral energy source is made available for the production of electricity. Secondly, grass clippings are diverted from the landfill. Thirdly, residential hot water is produced using a smaller carbon footprint. Fourth, additional economic activity and jobs are created for the economy.

In addition, waste heat from factories, power plants, furnaces and the like can and should be used to provide space heating, hot water, and steam through the use of heat exchangers. For example, waste heat from a gas furnace or gas water heater could be used to preheat water to the water heater.

### Production of Ethanol at the Electricity Generation Site

Another proposal consists of building an endothermic process such as an ethanol fermentation capability at the electricity generation site in addition to the deployment of the invention, a hybrid clean energy unit. In so doing, some percentage of heat from electricity generation would be needed by the ethanol production during the entire year, and the amount of fossil fuel needed for heat energy would be reduced through the use of the invention, a hybrid clean energy unit. In the cooler months of the year, ethanol production may need to be reduced so that heat energy could be redirected to the invention, a hybrid clean energy unit. Over the course of the year, the electricity generation would occur as before except that less fossil fuel would be required and significantly less waste heat would be released to the environment due to the addition of the ethanol production capability and the hybrid clean energy unit. In addition, the production of ethanol and food would be available for sale to the marketplace.

The third method consists of adding only an endothermic process capability, such as an ethanol production capability to the electricity generation site. In this scenario, waste heat from the electricity generation would be used to drive the fermentation process. In so doing, the waste heat from electricity generation is reduced, and a valuable liquid fuel is produced.

Another proposal consists of the deployment of Energy Storage and Electrical Generation Equipment on the electricity produced from Hybrid Clean Energy Unit collocated near a traditional electrical generation facility.

Another proposal consists of the deployment of an Energy Storage and Electrical Generation Equipment for the electricity produced by a traditional electrical generation facility.

It is important to note that a variety of materials can be used for ethanol fermentation. Some crops may be used for ethanol fermentation. The waste from some crops may be used for ethanol fermentation. Food waste and paper waste may be used for ethanol fermentation instead of being dumped in a landfill. Cardboard and wood waste may be used for ethanol fermentation. Grass clippings may be used for ethanol fermentation. Biomass may be used for ethanol fermentation, and FIG. 3 shows how solar energy may be used to crack the biomass; this capability is quite significant because the use of solar energy to crack biomass vastly improves the sustainability of the process of making ethanol from biomass. Without this capability, the use of biomass is rather limited due to the fact that more fossil fuel goes into making ethanol than is ultimately derived from the ethanol upon its combustion using the current state of the art.

In light of this new role for paper and plastic to be transformed into fuel, it behooves the manufacturers of paper and plastic to produce these materials in a manner that makes them the most conducive to the chemical processes that will transform them into fuel. For instance, plastic items may have no need for some of the additives presently utilized in their manufacture that would impair the potential of these plastic items to be upgraded into fuel.

Some of the materials mentioned in the previous paragraph are recyclable. The sad fact of the matter is that only a fraction of these recyclable materials actually get recycled; their destiny is the landfill if they are not recycled. Therefore, the second and third methods provide a useful, dependable need for some recycled products that would otherwise be dumped in a landfill.

### Residential Production of Ethanol

As described above, the solar thermal collection embodiments 10, 11, can collect solar energy for direct use as heat or as electricity. The residence and its inhabitants also generate significant waste biomass, including food waste, grass clippings, etc. There are clear situations where a liquid fuel is desirable. One implementation of this involves an integration of energy output from the solar thermal collection embodiment 10, 11, with a residential scale ethanol unit, as shown schematically in FIG. 3. The method for small residential production of alcohol has presented a number of challenges with respect to sustainability, economics, and feasibility as well as safety.

Cellulosic ethanol has received a great deal of attention because of the inherent sustainability benefits. However, this approach requires too much energy to crack the biomass using the current state of the art to be feasible at the residential level. In so doing, the sustainability of the production of ethanol from Biomass is vastly improved because sunlight is now being used for a highly energy intensive step of the process, instead of a fossil fuel using the current state of the art. In so doing, the input energy to the process is vastly reduced, so the entire process now has much more favorable economics.

In the fermentation process 200 of FIG. 3, biomass from a variety of sources is brought together and prepared for processing in a biomass preparation step 202. In a batchwise manner, the biomass is introduced into fermentation vats with water and a fermentation medium, typically yeast. The fermentation process 204 generates, as is well known, an ethanolic aqueous solution, carbon dioxide and solid waste. The carbon dioxide is separated off as it is generated, and it can be utilized in one or more greenhouse units that comprise a part of the sustainable energy residential unit. The liquid can be drained or pumped off to a conventional distillation unit 206 and the solids remaining in the fermentor can be moved to a unit 208 dedicated to solids repurposing. The solids will contain, among other things, yeast biomass. Depending on composition, the solids may provide a food source for livestock or domestic animals in some cases and, upon being dried, typically using heat from the solar thermal collection unit, may be a combustion fuel to provide heat or to raise steam, using conventional processes. Solids repurposing in unit 208 may be especially efficient when the biomass feed to the fermentation unit 204 from the biomass preparation unit 202 is of a uniform nature. As an example, if the biomass is a good starch/sugar source, such as grain, the solids remaining will be very suitable for feeding poultry, swine or livestock. Waste heat, from a variety of sources, is useful for drying the expended grain coming out of the fermentor.

At the present time, residential operation of a distillation unit 206 may run afoul of local, state or federal regulations. A simple solution to this could be provided by requiring that the ethanol be rendered unfit for human consumption by addition of a chemical agent. Such agents are well known. When ethanol is used as a motor fuel, for example, methanol is the preferred chemical. In other situations, another agent that provides an objectionable taste to the ethanol can be used. This step is important, because, depending on the biomass used, the fermentation process may already have sufficient methanol in it to render the fermentate unfit for consumption. After being distilled, the ethanol may be moved to a fuel storage unit 210 for subsequent use, which may include use as a stationary fuel or as a mobile fuel. In an alternative to distillation, known membrane separation technology may be used to concentrate the ethanol.

It is clear that the cogeneration and fermentation concepts taught herein are easily scalable. An installation can be done on a single home or building, two or three or more single homes or buildings, or a small or big warehouse or store, or a small or big factory, or a small or big commercial or residential building. The possibilities are endless for possible applications of these inventions.

It is also important to note that an Energy Storage and Electrical Generation System may be added to any of the foregoing inventions to provide an electrical power back up system to the home, business, factory, commercial building, hospital, and/or warehouse in the event that the primary source of power experiences a shutdown. The Energy Storage and Electrical Generation System is highly scalable and very flexible for any application. In addition, the material used for this equipment is air. Air is reusable, highly sustainable, environmentally-friendly, and available in more than adequate supply throughout the word at no cost.

The use of sugar is very suitable for residential production of ethanol, yet the economics of ethanol present quite a challenge. Since it takes roughly 10-14 pounds of sugar to produce a gallon of ethanol, one raw material accounts for $2.00-$2.80 per gallon of ethanol, and nothing else has been purchased. At the time of this writing, gasoline is $2.00-$2.25 per gallon. So, the economics of using sugar certainly are not promising in the United States. However, the economics of sugar might have better viability in Central and South America if ethanol fermentation activity increased. Perhaps a bigger demand for sugar in Central and South America might provide farmers in these areas with another cash crop instead of plants used to produce illicit drugs. In the United States, sugar presents a problem. We essentially exchange our current dependence on foreign sources of oil for dependence on foreign sources of sugar.

The next idea that comes to mind is corn. Once again, the economics is dicey because 2.8 gallons of ethanol require one bushel of corn. Corn is currently priced at $3.89 per bushel. So, the cost of one raw material is $1.39 per gallon of ethanol, and nothing else has been purchased. In addition, the use of corn raises the issue of food verses fuel. Furthermore, the use of any other crop also raises the food verses fuel debate.

At this point, the idea of waste streams came to mind. In particular, the use of alcohol waste, soda drink waste, and food waste were discussed in several online websites. The use of these waste streams presents a number of benefits. First of all, their use is truly aligned with the cause of sustainability. Secondly, the economics and feasibility are favorable. Third, the use of these items for residential ethanol diverts these waste streams from a landfill where they will eventually be transformed into methane, which simply pollutes the air. Safety is the final issue. Any use of a distillation process to separate alcohol from water presents risk; however, this risk can be addressed with the appropriate use of process hazard analysis as well as some common sense. Finally, and perhaps sadly, food waste is in abundant supply in the United States. Approximately, “30-40% of the food supply is wasted,” according to worldfoodusa.org, “which amounts to 20 pounds of food per person per month.”

The process layout for residential bioethanol is presented in FIG. 3. The equipment needed is basic. The overarching concept is to employ an operator to do the hands-on work at least 5 days per week and to leave the resident with less than one hour per week on Saturday and Sunday. The operator would pick up food waste at least 6 days per week, run the process Monday thru Friday, pick up ethanol product, perform maintenance, and assure the process is running. Additional automation opportunities are available; however, the cost and complexity rises as well. Alcohol waste would be ready for distillation, soda drink waste would be ready for fermentation, and starchy food waste would require hydrolysis then fermentation and then distillation.

Implementing a residential ethanol production system of this type would require several items of equipment, including for illustrative purposes, a distillation apparatus, drying equipment (preferably, solar powered) to prepare the distillers dried grains, open-head drums with lids to prepare mash, air locks and agitators, gas collection system for collecting carbon dioxide released during fermentation, automated inoculators for yeast, and a reflux still, preferably heated by waste heat from cogeneration.

This system would also require capacity to deliver and remove grain products.

To the extent that this system is using waste food products from restaurants, bars, grocery stores and other food waste pickup sites, there will be a need to employ and train waste stream collectors on desired and undesired food waste as well as which drum to use for each waste stream, preferably avoiding high salt food waste.

During implementation, food waste would be collected and sent through size reduction to provide a feed stock of a consistent nature.

Once delivered to a residential site, the new or “virgin” food waste would be treated to saccharify it through the hydrolysis cycle and inoculated with yeast for fermentation. From at least this point, the process should be automated to the fullest extent possible. After about twelve hours, the inoculated mash should be ready for fermentation. Drums containing the mash can be rotated through the system, with the drums being cleaned prior to re-use. Alcohol content of the mash can be automatically monitored, or a designated amount of time for fermentation may be selected based on the rate of conversion and the schedule of process operators. Distillers Dried grain can be shipped out for composting or for sale as animal feed or fertilizer.

Fermented mash is then distilled, and, if desired, can be processed to an even higher alcohol content using molecular sieves, which can be regenerated as needed.

To the extent possible, waste heat from trucks used in the delivery and pick up process can be used for drying waste food, distillers grain and for regenerating the molecular sieves.

One of the avenues under consideration for cleaner emissions from cars has been the replacement of the use of gasoline with the use of natural gas. Natural gas has a number of benefits over gasoline as a transportation fuel; however, its implementation into gasoline burning societies has been very slow. People will buy natural gas vehicles when natural gas fueling becomes available; however, fueling stations will become available when natural gas vehicles are built and sold. Dual fuel gasoline/natural gas vehicles present an opportunity to bridge the transition to natural gas, yet such conversions have not become popular with the general public. Even with such conversions, the compression of natural gas to compressed natural gas remains an issue.

With the carbon dioxide levels and the melting of glaciers discussed earlier, a need has arisen to fuel vehicles with natural gas sustainably without the vehicle challenges, the natural gas compression challenges, and the natural gas fueling infrastructure challenges.

### Storage of Excess Energy

In many systems for where solar power or wind power are harvested, the instantaneous production of energy rarely matches the instantaneous energy use. When insufficient power is produced, the user needs to import energy, typically from the electrical grid; and when excess power is produced, the excess power can be exported to the electrical grid. In this latter case, it is necessary to provide the excess power as a “clean” alternating current electrical signal that has a frequency and voltage that is synchronized with the grid signal. In many cases, the excess power may be in the form of heat energy or, if stored in a traditional battery, in the form of direct current.

Further, the production of large storage batteries may be highly damaging to the environment, due to the elements such as cobalt and lithium that are used to produce the batteries.

For at least these reasons, preferred embodiments of the present invention include a system which the inventor prefers to call the “air motor” or “air battery”, as shown in schematic view in FIG. 4. In this system 300, the equipment provided includes a volume reduction unit 302 and an expander 304, preferably in the nature of a turbine. One or more storage units 306 are also provided. Input energy 308 may be generated in a variety of forms, but it is typically excess energy as may be generated by a solar thermal apparatus, such as units 10, 11 of FIG. 1. Input energy 308 may also be provided by ethanol produced fermentation 204 of FIG. 3, by combustion of solids from solids repurposing 208 of FIG. 3, and other sources. If input energy 308 is used to operate the volume reduction unit 302, a compressible fluid, such as air, may be compressed and stored in one of the storage units 306 until such time that energy is needed. When that occurs, the compressed fluid is sent to expander 304, where the expansion of the fluid operates a generator, providing output energy 310. The expanded fluid, especially when it is air, can be exhausted to the atmosphere. Many embodiments of the volume reduction unit 302 would be conventional compressors, bur, alternatively, known technology maybe used to liquefy the air and, in some cases, to separate the air into component gases, for particular use. Also, and while the use of air as the operative fluid may be preferred, due to the availability of supply and the ability to exhaust the gas without environmental consequence, other gases, as separated from air, may be used. However, the primary advantage of using air is that the process remains “open” at each end, that is, there is no need to maintain a supply of the uncompressed gas, which would entail a significant capital investment.

Output energy 310 may be utilized in a number of ways. However, one clear use would be to use the energy as an input energy to the distillation unit 206 of FIG. 3.

### Use of Fossil Fuels in a Typical Suburban Household

The inventor is a typical resident of the mid-western United States. Our household is in a suburban neighborhood. The inventor lives in an approximately 2600 square foot house, with five residents, four vehicles, and four car drivers. The residence has electric service for household electricity needs and air conditioning, and natural gas service for the furnace, hot water, and a fireplace insert. At the time of installation, high efficiency HVAC equipment and appliances were purchased and installed.

The inventor proposes a method for sustainably replacing gasoline usage with natural gas. The method involves installation of a 1-9 kW household cogeneration unit powered by natural gas, although the cogeneration unit could be fueled by a number of other fuels. Waste heat from the cogeneration unit is used for hot water and space heat in the winter months, and this waste heat is used in the summer months to provide the needed heat energy to ferment sugar or an equivalent raw material into ethanol. An underground tank may be needed to store the ethanol for subsequent use depending on the location of the home and depending on the arrangements made with any operating personnel. Instead of using gasoline powered cars, the household will use plug-in hybrids equipped with supplemental batteries and with a combustion engine capable of running on gasoline or E95. In addition, the batteries in the cars will be set up in interchangeable modular units so that each car is set up with an appropriate electric storage capacity for its respective daily commute. Furthermore, the batteries in these vehicles would be located in the trunk for the flexible use of the vehicle.

During the regular schedule of work and activities, the car would be powered primarily by electricity produced from natural gas and the ethanol produced. During a vacation or a big trip, the batteries in the trunk could be removed, and the car would become a hybrid powered by E95 and/or gasoline. Depending on battery cost and the cogeneration unit cost, high voltage could be generated for fast recharging of the minimal amount of batteries, or regular voltage could be generated for a slower recharge of perhaps a greater number of batteries. Using regular voltage, cars would either need to recharge for a longer period of time, and/or battery modules would have to be switched out among the vehicles, and/or additional battery modules would have to be employed. A major benefit of using largely electric power for commuting in a car is the reduction of car exhaust in urban areas which will yield a significant improvement in air quality once a high level of adoption of this invention occurs. Another design aspect of the inventive hybrid vehicle has arisen. Many of the mining techniques used for various chemicals in the production of batteries have significant, serious and deleterious long-term effects on the environment. Thus, the use of batteries in plug-in hybrids has negative environmental impacts that are perhaps unanticipated and/or unknown by many potential car buyers.

To address this problem, another power system for the inventive hybrid vehicles is needed. The inventor proposes the use of a liquid nitrogen or liquid storage and air drive system instead of, or in addition to, the batteries. The liquid nitrogen or liquid air would be heated by exhaust heat from the conventional fossil fuel or alcohol engine. Upon heating, the liquid nitrogen or liquid air would vaporize, and a very significant volume expansion would occur. The volume expansion would provide energy to power either a traditional air motor, or perhaps a turbine which could be located in the rear portion of the vehicle. The horsepower from this nitrogen-driven or air-driven engine may be used to provide all-wheel drive to the car using a viscous coupling drive train, which, I believe, has been used quite extensively by Subaru. However, other methods to transmit this energy to the wheels of the car may be utilized. A second alternative arises through the coupling of a generator to the nitrogen-driven or air-driven motor. The generator would produce a supply of electricity that would power the electric engine that is used in the traditional hybrid vehicle architecture; this approach allows the automaker to take advantage of recent developments in the electrification of automobiles while still allowing the reduction of the size of electric battery banks, which have proven to be a significant design issue in these vehicles due to the cost, weight, limited life, and the negative environmental impact of these batteries.

The vaporized nitrogen or vaporized air could be safely emitted to the atmosphere as it originated from the atmosphere; the nitrogen or air is thereby used as an environmentally friendly energy carrier. Alternatively, the liquid air or liquid nitrogen may also be co-fed to a traditional combustion engine, similar to the practice of adding liquid water to aircraft engines for additional range during World War II.

It is important to note that the methods noted above for using nitrogen and air as an energy storage medium would be very suitable for providing a power storage capability for renewable energy sources. Excess electric power could be directed to cryogenic air separators or liquefiers that would produce cryogenic liquids that could be used later as the need arises to produce electricity. In California, the use of cryogenic air separators or liquefiers would help to deal with the problem of electricity oversupply that has arisen during the daytime when solar electricity plants are currently able to provide excess quantities of electricity that are not currently needed in the marketplace.

It is very important to note that the ideal siting location for cryogenic air separators would be fossil fuel electricity generation sites. The liquid nitrogen and other valuable constituents of the atmosphere could be transported to the marketplace; however, liquid oxygen could be used at the electricity generation site to combust fossil fuels. If a given electric facility were using only oxygen to combust fossil fuels, the emissions from this facility would be almost entirely carbon dioxide arid water. An emission stream containing mostly carbon dioxide and water could be separated by simply condensing the water vapor from the carbon dioxide or absorbing the water in media from the carbon dioxide.

The stream of carbon dioxide could then be used to make a variety of products, including but not limited to, all kinds of blown plastics, urethanes, rubbers, and acrylics as well as fire extinguishers using carbon dioxide and carbonated sodas. In addition, the carbon dioxide and water stream could be fed, in part, to a hybrid clean energy unit.

As an alternative, cryogenic air separators or liquefiers could be made at residential scale so that households that implement, or do not implement, cogeneration equipment could also be able to generate their own liquid nitrogen or liquid air at their residence for their vehicles. As such, households that adopt this technology would be able to produce their own electricity, liquid nitrogen and/or liquid air, ethanol, and/or space heating and hot water. A number of supply options will allow competition to occur in the marketplace and will help to ensure that customers are not captive to only a single supply source.

In many embodiments of the invention, the interchangeable battery modules for inventive hybrid vehicles would be useful to power other items around the house, such as a back-up battery powered sump pump, a lawn mower, a snow thrower, a blower, a line trimmer, a pruning saw, a trouble lamp, a drill, or a power saw

It is important to note that electric motors last a long time. In addition, electric motors do not require as much service, and are easier to get started. Finally, many small, fossil fuel engines tend to produce dirty emissions, and they also tend to be noisy.

Alternatively, a liquid-nitrogen or liquid air storage and electrical generation system could be wired into a circuit to provide back-up power to mission-critical equipment in the household, office, factory, small or big business, farm, hospital and/or warehouse.

In addition, a storage tank and a circulation pump for hot process water and/or a heat transfer fluid would be needed to buffer the exhaust heat from the cogeneration unit to the household heat energy needs. Finally, a control system would be needed to operate all of this equipment in an orderly manner. It is important to note that the house would still have a furnace and hot water tank fired by natural gas should the need arise. Also, the house would have electric air conditioning either powered by the cogeneration unit or the electric utility. Moreover, the house would have an electric connection from the utility that allows electricity to flow from the grid to the house and from the house to the grid. This electric connection would provide additional energy to the house for peak needs, and it would avoid the need for a household energy storage unit. However, if such a connection is not available, a household energy storage unit could be included. In addition, the electric cogeneration unit would be equipped to sense a power outage and provide essential energy to the household during a power outage.

Admittedly, this household has been equipped with a great deal of equipment upgrades. However, the vehicles in the household are now fueled by natural gas in a sustainable manner. In addition, the attached spreadsheet details the fact that the energy use for the household using this method has been reduced by approximately a factor of 2, and the waste heat to the environment has been reduced by the heat that would be produced by burning in excess of 200 mcf of natural gas. Each mcf of natural gas yields 1,000,000 Btu, so that is a substantial reduction of waste heat. This method could be adopted by apartments, condominiums, office buildings, and other facilities to yield similar results.

### Economics Discussion

For illustrative purposes, the inventor has prepared an estimate of the economics for applying this concept to a family of five persons, residing in a 2600 sq ft house. Of the five residents, four of the residents drive a vehicle.

Current annual energy cost for this residence is estimated at $10,010, based on:
electricity for air conditioning at $500;
natural gas and electricity (for powering blowers) for a furnace at $700;
miscellaneous electrical usage at $1,900;
hot water heater (natural gas) at $300,
fuel for the four vehicles at $3,600;
heating and cooling maintenance at $256; and
vehicle maintenance at $2754.

Under an application of the concept, annual energy costs would be estimated as:
electricity for air conditioning at $95;
natural gas and electricity (for powering blowers) for a furnace at $502;
miscellaneous electrical usage at $906;
hot water heater (natural gas) at $0 (system eliminate),
fuel for the four vehicles at $1050;
heating and cooling maintenance at $256; and
vehicle maintenance at $2165.
   These costs total to $4974. However, implementation of a cogeneration system for the residence would be expected to add an annualized cost of ownership (as described in more detail below) of $5375, bringing the annualized costs to $10,349.

The reduction in fuel cost for the vehicles is based upon operating the vehicles on electricity generated by the cogeneration unit with an electrical charge equivalent to the estimated use of 6 gallons of gasoline per day for 240 days per year. As described below, the cost of conversion from gasoline to electric vehicles is included in the annual cogeneration cost.

The annual cost of ownership of the cogeneration unit is based on purchase cost, interest cost, maintenance cost and incremental costs. As described, the capital cost is estimated at $86,000, financed at 3% per year over 20 years, for a total cost of $137,600. Of this, $30,100 is depreciable, leaving $107,500 to be spread over 20 years, for the $5375 per year.

The $86,000 in capital cost breaks down into three costs: $51,000 to purchase, install and maintain the cogeneration system; $30,000 in incremental cost for the vehicle conversion and $5000 in incremental costs associated with the heating system. These latter costs include $3300 for a thermal storage system and a control system, $400 for a pump, $500 for heating duct coils and $800 for water heater tank coils.

According to the inventor's estimates, an indoor or covered swimming pool with a surface area of approximately 10 ft by 20 ft could use heat from the cogeneration unit to maintain the water temperature at about 20 F above ambient. A pool like this could be used by the residents from approximately late May to mid-September, depending upon the weather, if the residents preferred a pool instead of an ethanol production capability.

Another proposal consists of residential ethanol production driven at least in part by solar energy. In this proposal, ethanol fermentation would occur during the warm months of the year in the specific locale using previously discussed methods, and the thermal input to the fermentation would be provided by solar thermal equipment.

### Sustainable Air Conditioning and Sustainable Electricity Generation

As noted above, a complex of causes has resulted in global warming, otherwise known as climate change, that must be addressed properly sooner rather than later. It is easier to understand how waste heat emissions cause warming rather than a greenhouse effect. It is more difficult to understand how higher levels of carbon dioxide and other greenhouse gases in the atmosphere cause a greenhouse effect which leads to global warming. Yet, the term greenhouse effect has existed for a long, long time.

While carbon dioxide has received much of the attention, other gases have the potential to be of greater impact. Of particular note is the presence of a number of fluorinated hydrocarbons. Global warming potential (“GWP”) has been defined as the ratio of the time-integrated radiative forcing from the instantaneous release of 1 kg of a trace substance relative to that of 1 kg of a reference gas. In the table below, “HFC-134a” is 1,1,1,2 tetrafluoroethane (CH₂FCF₃), also known as FREON 134a, “HFC-23” is fluoroform (CFH₃), “HFC 125” is pentafluoroethane (C₂HF₅), “PFC 14” is tetrafluoromethane (CF₄), “PFC 116” is hexafluoromethane (C₂F₆) and SF6 is sulfur hexafluoride (SF₆). While many of these compounds are used as refrigerants, SF6 finds its most common application as a dielectric medium. To demonstrate the effect of these gases, data from various sources, including www.gov.uk provide the following picture, in tabular form:

| Fluorinated HC | % of total FHCs | GWP | GWP effect |
|---|---|---|---|
| HFC-134a | 58 | 1430 | 829.4 |
| HFC-23 | 5 | 14800 | 740 |
| HFC-125 | 9 | 3500 | 315 |
| HFC-152a | 20 | 124 | 24.8 |
| PFC-14 | 5 | 9300 | 465 |
| PFC-116 | 1 | 12200 | 122 |
| SF6 | 3 | 22800 | 684 |
| Total FHCs | 101 | | 3180.2 |

These data can be compared in the aggregate with other common greenhouse gases to determine the overall effects of their presence in the atmosphere, as follows:

| Greenhouse gas | % of total | GWP | Life in Atmosphere |
|---|---|---|---|
| Carbon dioxide | 82 | 1 | 100 Years |
| Methane | 10 | 36 | 12 Years |
| Nitrogen oxides | 5 | 298 | 120 Years |
| Total FHCs | 3 | 3180.2 | Weeks to 50,000 |
| | | | Years |
| Total greenhouse | 100. | | |
| gas | | | |

In view of these data, FHC's (Halocarbons) may obviously warrant much more emphasis and action.

Various FHCs were banned by the Montreal Protocol due to the deleterious effect of these chemical agents on the ozone layer. Based on the information in the second table, another ban may be warranted. However, the current generation of FHCs is an essential component for electrical air conditioning. Therefore, a ban of these chemicals would essentially amount to a ban on electric air conditioning, a most unwelcome development for many locales in the world. Another alternative is needed, and another alternative is available. Cryogenic air separation units will be able to separate air into any desired constituent combinations. Liquid carbon dioxide, alone or in combination with any mixture of noble gases may prove to be a suitable refrigerant for air conditioning units. Another alternative lies in simply using a suitable combination of noble gases with or without nitrogen. Yet another alternative lies in using a suitable combination of noble gases with nitrogen and with or without carbon dioxide. Liquid oxygen may be used with any of these combinations; however, proper precautions need to be taken because oxygen is a powerful oxidizer. In any case, the foregoing chemicals offer a much more sustainable and environmental profile to the environment than the current generation of fluorinated hydrocarbons. In addition, any of the above combinations will prove to be much more sustainable and environmentally friendly than SF6, which has a GWP of 22,800 (the highest GWP in the first table).

Until yet another generation of fluorinated hydrocarbons (or other suitable refrigerant) is developed and proven to be safe for the environment, absorption chiller technology offers another feasible alternative to electric air conditioners which use these problematical fluorinated hydrocarbons. Absorption chiller technology is admittedly less energy efficient than electric air conditioning; however, fluorinated hydrocarbons are no longer required with absorption chiller technology. Absorption chillers can be economical because they can be driven by natural gas or any fossil fuel for that matter as well as heat from a renewable energy source. The sustainability of absorption chiller technology can be quite significantly improved through the use of solar thermal collection with a heat storage capability rather than only using natural gas to drive these units. Such an arrangement is particularly favorable because the highest need for heat for an absorption chiller occurs when the outside temperature is the highest, and the highest outdoor temperature would likely occur during the day when the sun is out. Therefore, a solar thermal collector will typically be most productive during the hottest intervals of the day when the most air conditioning is required from an absorption chiller unit.

Initially, it might appear that carbon dioxide would be a poor alternative to halocarbons as a refrigerant, since the objective is to reduce greenhouse effect. However, since carbon dioxide has a much lower GWP, is readily available in the environment and, when utilized as a refrigerant, is effectively sequestered, it becomes a much more attractive alternative, when it is contained in a closed-loop system.

It is straightforward to employ absorption chillers in residences, offices, apartments, condominiums, and commercial buildings. The adoption of absorption chiller technology in automobiles may present a problem due to the weight of these units even though a source of heat to drive these chillers is readily available in the automobile in the form of the exhaust heat from the engine.

Another alternative is available through the use of a tank of liquefied gas in automobiles that would be used to bring cars to a comfortable temperature in the warmer months. Essentially, the liquefied gas would cool the air in the passenger compartment using a heat exchanger. When the liquefied gas has provided cooling to the automotive compartment, it would boil, and the equipment would be designed to exhaust the vaporized gas to the environment.

Ideally, the liquified gas tank in the car would be sized so that it could be filled at the gas station when gasoline is needed. As an alternative, liquified gas could be delivered to homes and workplaces, and could be refilled at those locations more frequently allowing a smaller tank to be used in the vehicle. Another alternative lies in using liquified gas for smaller air conditioning loads in homes, apartments, condominiums and offices. Yet another alternative lies in the deployment of a residential size cryogenic air separation unit capable of providing at least liquid nitrogen as well as other desired liquid gases from the air.

If liquid nitrogen were adopted for cooling automobiles and/or smaller real estate properties, the ideal location for the cryogenic air separation units would be near electricity generation facilities using fossil fuel. With this siting arrangement, the liquid oxygen that is also produced by the process could be utilized by the electric generator for cleaner combustion of coal, biomass, fuel oils, and perhaps even natural gas.

The use of liquid oxygen for fossil fuel combustion may seem unnecessary; however, its use offers a number of advantages. First, nitrogen oxides present an air pollution issue. If emissions of fluorinated hydrocarbons and nitrogen oxide were dramatically reduced, air quality could be improved. Secondly, exhaust emissions from a fossil fuel facility would be limited to largely carbon dioxide and water vapor. The water vapor could be easily separated from the carbon dioxide. The carbon dioxide could be used for productive purposes such as enhanced oil recovery or perhaps as a blowing agent in insulation and/or foam. The foams could be made from the following new or waste materials: rubber, plastics, urethanes, acrylics, polystyrene, polyvinyl chloride, polyethylene, polypropylene, water bottles, etc. The water vapor could be condensed in a cooling tower, and then it could be distilled, if necessary, using waste heat from the electricity generation operation so that it could be brought up to a level of integrity that would be considered potable and/or acceptable.

When these new innovations described above are deployed along with the hybrid clean energy unit for electrical generation and the alternate fueling configurations for vehicles as well as scalable Energy Storage and Electrical Generation Systems, our future is much brighter from the standpoint of sustainability. The full impact of all of these inventions will address the nexus of energy, food, water, clean air, climate change, space heating, hot water needs, energy storage, transportation, and air conditioning, as well as space travel potentially, which collectively would require a smaller carbon footprint and would have a much more favorable profile of negative environmental impacts than the technology currently in use.

Having shown and described a preferred embodiment of the invention, those skilled in the art will realize that many variations and modifications may be made to affect the described invention and still be within the scope of the claimed invention. Thus, many of the elements indicated above may be altered or replaced by different elements which will provide the same result and fall within the spirit of the claimed invention. It is the intention, therefore, to limit the invention only as indicated by the scope of the claims.

1. A system for integrating energy usage, comprising:
a system for capturing solar energy;
a system for fermenting biomass and concentrating the fermentate, generating carbon dioxide and ethanol;
a system for storing excess energy for subsequent release; and
a system for growing biomass;
wherein solar energy captured is used as heat and electrical power, with any excess energy being stored in the system for storing excess energy, ethanol is used as a fuel, carbon dioxide is provided to the system for growing biomass, and instantaneous energy deficiencies are reduced by releasing stored excess energy.

2. The system of claim 1, wherein:
the system for capturing solar energy comprises a thermal transfer fluid circulated in tubes.

3. The system of claim 2, wherein:
the system for capturing solar energy further comprises reflectors to focus the solar energy onto the tubes

4. The system of claim 1, wherein:
the system for capturing solar energy comprises photovoltaic cells generating electrical energy.

5. The system of claim 1, wherein:
the system for capturing solar energy is arranged inside an attic space of an inhabitable dwelling.

6. The system of claim 1, wherein:
the system for storing excess energy comprises:
a compressor, powered by excess energy from the system for capturing solar energy solar to compress a fluid;
at least one storage means for containing the compressed fluid;
an expander for expanding compressed fluid from the at least one storage means, power generated by the expander used to reduce instantaneous energy deficiencies in the system.

7. The system of claim 1, wherein:
the system for storing excess energy comprises:
a gas liquefaction unit, powered by excess energy from the system for capturing solar energy solar to compress a fluid;
at least one storage means for containing the compressed fluid;
an expander for expanding compressed fluid from the at least one storage means, power generated by the expander used to reduce instantaneous energy deficiencies in the system.

8. The system of claim 6 or claim 7, wherein:
the fluid compressed is air.

9. The system of claim 2, wherein:
the system for capturing solar energy is arranged inside an attic space of an inhabitable dwelling.

10. The system of claim 3, wherein:
the system for capturing solar energy is arranged inside an attic space of an inhabitable dwelling.

11. The system of claim 4, wherein:
the system for capturing solar energy is arranged inside an attic space of an inhabitable dwelling.

12. The system of claim 7, wherein:
the fluid compressed is air.
